(19) United States
(12) Patent Application Publication
(10) Pub. No.: US 2017/0360700 A1
(43) Pub. Date: Dec. 21, 2017

(54) Flavored Non-Fructose Compositions and Methods for Negating Dehydration

(71) Applicant: H2ORS, Inc., Orinda, CA (US)

(72) Inventors: Candace Fleming, Orinda, CA (US); Christopher Shaw, Woodside, CA (US)

(73) Assignee: H2ORS, Inc., Orinda, CA (US)

(21) Appl. No.: 14/316,706

(22) Filed: Jun. 26, 2014

Related U.S. Application Data
(60) Provisional application No. 61/957,199, filed on Jun. 26, 2013.

(51) Int. Cl. A61K 9/00; A23L 2/38; A23L 29/30; A23L 33/16; A61K 31/7004; A23L 2/56

(52) U.S. Cl. CPC A61K 9/0095; A61K 31/7004; A23L 2/56; A23L 29/30; A23L 33/16; A23L 2/38

(57) ABSTRACT

Embodiments of the present invention are directed to ingredients, formulations, methods of manufacture and use of an oral rehydration solution (“ORS”) in the prevention and treatment of dehydration in humans. More specifically, invention embodiments relate to flavored electrolyte drink mixtures that relieve the dehydrating effects caused by travel, exercise, over-indulgence of toxic substances, and various illnesses in individual subjects. An ORS embodiment of the present invention restores fluid and electrolyte balance in humans as fast and as effectively as IV therapy, and thereby reduces the incidence of dehydration in human populations as well as rehydrating individuals in circumstances where IV therapy is not practical or wanted. The disclosed ORS therapies and packs may be used in conjunction with IV therapy as a multi-part strategy to allow hospitals to lower patient re-admission rates, lower staffing needs, give outpatient self-therapy to treatments prescribed outside of the hospital environment, and thus lower treatment and process costs while not reducing system or treatment efficacy.

### CROSS-REFERENCE TO RELATED APPLICATION

Applicant claims priority and all benefits of U.S. Provisional Patent Application Ser. No. 61/226,263, filed Jul. 16, 2009, which is hereby incorporated by reference in its entirety.

### DESCRIPTION OF THE INVENTION

Improved low osmolarity, flavorful hydration formulations and therapies are very much needed and in demand in both personal and healthcare-related environments. It should be understood that low osmolarity, flavorful solutions, beverages and other beverage products in accordance with this disclosure may have any of numerous different specific formulations or constitutions. The formulation of compositions in accordance with this disclosure can vary to a certain extent, depending upon such factors as the product's intended market segment, desired nutritional characteristics, flavor profile and the like. For example, it will generally be an option to add further ingredients to the formulation of a particular embodiment, including the formulation described below. Additional (i.e., more and/or other) sweeteners may be added, flavorings, vitamins, colorants, fruit products, masking or muting agents and the like, and/or flavor enhancers typically can be added to any such formulations to vary the taste, hydration characteristics, nutritional values, and so forth. Based on the guidance provided herein, formulating such other products will be well within the ability of one skilled in the art of formulating low osmolarity oral rehydration solutions (“ORS”) and products. Such products are also covered by the scope of this invention. Exemplary embodiments of ORS used in developing countries and disaster relief zones is taught in the WHO disclosures of Bhan M. K., et al., Clinical trials of improved oral rehydration salt formulations: a review, Bulletin World Health Organ, 1994;72(6):945-55, which is hereby incorporated by reference in its entirety for background and examples of knowledge in the art. To date, while some ORS formulations have been used in hospital and personal settings, those used all suffer from flavor-related short-comings of their respective formulations.

Specifically, flavored dextrose formulations for IV adjunct therapy is an improvement to the formulations and processes disclosed in Bhan. Further, the WHO solutions teach total osmolarity values of 245 mOsm/L, and only further teach combined analysis of many studies of what the group refers to as “reduced osmolarity” solutions, with total osmolarity values between 210-268 mOsm/L.

Embodiments of the present disclosure are directed to ingredients, formulations, methods of manufacture and use of pleasant-flavored low osmolarity oral rehydration solutions to be used for the prevention and treatment of dehydration in humans. More specifically, some formulations relate to electrolyte drink mixtures of a liquid and powder colloid that relieve the dehydrating effects caused by various illnesses, exercise, over-indulgence of toxic substances, and travel in individual subjects. The liquid may be water, and the water may be naturally derived or manufactured on-site. One example of in situ manufactured water is taught in U.S. patent publication ser. No.: US 2008/0135495 A1, which is incorporated by reference herein in its entirety.

An ORS embodiment of the present invention restores fluid and electrolyte balance in humans as fast and as effectively as intravenous (“IV”) hydration therapy, and thereby reduces the incidence of dehydration in human populations as well as rehydrating individuals in circumstances where IV therapy is not practical or wanted. Alternatively, ORS therapy may be used in conjunction with IV therapy as a multi-part strategy to allow hospitals to lower patient re-admission rates, lower staffing needs, give outpatient self-therapy to treatments prescribed outside of the hospital environment, and thus lower treatment and process costs while not reducing system or treatment efficacy.

This disclosure also allows for an IV treatment alternative for hospitalized patients, in addition to individuals who would prefer to avoid needles for any number of reasons. In addition to oral delivery of the compositions, fluid delivery devices known in the art may also be used, as contemplated by the inventors, for use with individuals who are too weak or incapacitated to hydrate without assistance.

As is understood in the art, low osmolarity formulations are typically bitter, tart, or in other ways unpleasant or having a disagreeable flavor typically associated with rehydration formulations. The present invention, though a number of flavorful additive combinations, and processes, provide a choice of taste improvements over the art. Exemplary flavors include, but are not limited to, lemon, cherry, strawberry, banana, and vanilla. Exemplary compositions and methods related to flavor engineering known in the art are disclosed in publication US 2008/0108710 A1 to Prakash et al., dated May 8, 2008, the disclosure of which is incorporated herein by reference in its entirety.

The terms “therapy” or “treatment” as used herein are intended to refer to use, treating, prophylaxis and/or suppression of dehydration through the use of fructose-free and flavored formulations that are administered within a low osmolarity oral composition. The treatments may be used to help patents suffering from dehydration caused by, but not limited to, chemo or radiation therapy, diarrhea and bowel-related conditions, kidney illness, environmental over-exposure and diabetes. Treatments are intended to include hospital-based IV therapy combined with hospital-based or non-hospital based low osmolarity oral therapy. This allows patients more freedom of routine, and ensures a higher compliance rate for home-based patients who started their treatment in the hospital setting. Alternatively, the inventors do contemplate fructose-related low-osmolarity solutions for the treatment of diabetes and individuals exposed to high temperature environments.

“Osmolarity” is defined herein as the number of osmoles of solute per kilogram of solvent (usually, not always, water), where one osmole is provided by each mole of ion charge. As known in the art, osmolarity is expressed as the concentration of osmotically active particles (i.e., osmoles) dissolved in 1 liter of water (1 mθsm/kg H2O at 38° C. is equivalent to an osmotic pressure of 19 mm Hg). So osmolarity as used herein refers to the number of solute particles dissolved in 1 liter of solution, the solution being water, or any other working solution. Solutes that may be added to the formulations so as to adjust the osmolarity of a formulation that includes, but not limited to, proteins, peptides, amino acids, non-metabolized polymers, vitamins, ions, salts (e.g., sodium or potassium salts), sugars (e.g. dextrose), metabolites, organic acids, lipids, etc. In one exemplary embodiment, the concentration of amino acids and salts, e.g., sodium and potassium salts (e.g., NaCl) in the formulations may be increased or deceased in order to achieve the desired osmolarity values set forth herein. When used herein, the abbreviation “mOsm” means “milliosmoles/L H2O”. The term “low osmolarity” or “low total osmolarity” as used herein is intended to refer to a total or combined solute osmolarity value of below 210 mOsm/L.

In exemplary embodiments, the compositions and methods provided herein are used to process or provide formulations that still have a positive flavor profile and have total osmolarity values in the range of between about 150 and 210 mOsm/L, or in alternative embodiments providing a formulation total osmolarity of under about 160, 180, 190, 200, 210 or 220 mOsm/L. One example of osmolarity-related teachings is found in U.S. Pat. No. 8,557,301 to Dolhun, dated October 15, 2013, which is hereby incorporated by reference in its entirety for background and examples of knowledge in the art. Dolhun, doe not adequately address the formulations and processes of the present embodiments taught and claimed herein. Dolhun teaches solutions with total osmolarity values of no lower then 230 mOsm/L solution.

Embodiments of the flavored low osmolarity formulations generally and broadly comprise zinc gluconate, dextrose, sodium chloride, potassium citrate, and sodium citrate to achieve both a low osmolarity and still function through activation of an individual's sodium glucose co-transport system. At the same time, the low osmolarity formulation solutes may be optimized to overcome the known unpleasant or disagreeable flavoring typically associated with rehydration formulations by adding any number or combination of compound solutes, including, but limited to, ascorbic acid, citric acid, natural flavoring additives and sucralose for flavor considerations and choice of taste improvements (exemplary flavors include, but are not limited to lemon, cherry, strawberry, banana , vanilla serving as a few examples contemplated by the inventors). The improved flavoring increases the chances of patient compliance with a hydration regiment in contrast with the typically disagreeable formulas now commercially available. This impressive and non-expected embodiment substantially improves the composition flavor, while the full ORS composition still maintains a low osmolarity as required for activation of the composition function through the treated individual's sodium-glucose co-transport system. Silicon dioxide or other drying/non-clumping excipients are included in the mixture to retard clumping of the composition in storage or when mixing with liquid such as water. Example I, below, teach one exemplary embodiment of the present invention.

### EXAMPLE I

Ingredients from one exemplary fructose-free embodiment of the present invention with suggested serving size of dry composition (without water or liquid added). The inventors contemplate other embodiments from the formula below.

| Composition Ingredient | Mg. input range per serving |
|---|---|
| Ascorbic acid | 150 mg-250 mg. |
| Zinc Gluconate | 5 mg-75 mg. |
| Dextrose | 4000 mg-6000 mg. |
| Sodium Chloride | 750 mg-1500 mg. |
| Potassium Chloride | 500 mg-1000 mg. |
| Sodium Citrate | 1200 mg-1600 mg. |
| Citric Acid | 1400 mg-2000 mg. |
| Natural Flavoring Additive | 3000 mg-4000 mg. |
| Sucralose | 30 mg-75 mg. |
| Anato or Beta Carotene | 5 mg-20 mg. |

Exemplary Dry Serving Size (Mg) would be within range of above ingredients.

### Breadth of Embodiments and Equlevants

In addition to the rehydration flavor formulations disclosed above, other non-fructose flavor enhancers or sweeteners are contemplated that relate to sugars, including a combination of sugars, such as glucose, sucrose, leucrose, trehalose, galactose, isomaltulose, dextrose, maltodextrin, corn syrup solids and/or glucooligosaccharides. These sweeteners are further intended to dissipate much of the bitter aftertaste associated with most low osmolarity oral rehydration solutions.

While this invention is satisfied by embodiments in many different forms, it is understood by those of skill in the art that the present disclosure is to be considered exemplary of the principles of the invention and is not intended to limit the invention to the embodiments illustrated or described. Accordingly, the inventors contemplate many related equivalent substitutions, and these inventions are not limited to the particular embodiments disclosed, but the embodiments are intended to cover all modifications that are within the spirit and scope of the invention as defined by the following appended claims.

What is claimed is:

1. A flavored low osmolarity fructose-free solution to be used by an individual in need of hydration, said use comprising: a) providing: i) a fine dry particle powder produced by mixing, grinding, crushing, or disintegrating one or more compounds including dextrose, sodium citrate, sodium chloride, potassium chloride, zinc gluconate, citric acid, ascorbic acid, sucralose, beta carotene, natural flavoring additive; and ii) silicon dioxide or at least one compound drying excipient; iii) mixing said dry powder into natural or manufactured water to form a solution; and b) further comprising orally administering a predetermined dose of said flavored low osmolarity solution to said individual in need of hydration in a manner such that hydration is achieved and low osmolarity is maintained.

2. A personal hydration pack, comprising the compound ingredients of claim 1.

3. A personal hydration pack of claim 2, wherein the compound ingredients of claim 1 are delivered to the individual orally or through a fluid delivery device.

4. A personal hydration kit given to an individual in need of hydration, and to compliment that individual's IV therapy, comprising a low osmolarity and flavored solution that contains no fructose.

5. A medical kit for on-site hydration for an individual in need, comprising the compound ingredients of claim 1.

6. The orally administered solution of claim 1, wherein the total osmolarity value is below 210 mOsm/L.
